(19) United States
(12) Patent Application Publication
(10) Pub. No.: US 2024/0027443 A1
(43) Pub. Date: Jan. 25, 2024

(54) PARTICLE AND PRODUCTION METHOD THEREOF

(71) Applicant: CANON KABUSHIKI KAISHA, Tokyo (JP)

(72) Inventors: KENGO KANAZAKI, Kanagawa (JP); FUMIO YAMAUCHI, Kanagawa (JP); NORISHIGE KAKEGAWA, Tokyo (JP); MASARU SUGITA, Tokyo (JP); RYO NATORI, Tokyo (JP); TAKESHI MURAKAMI, Kanagawa (JP)

(21) Appl. No.: 18/475,107

(22) Filed: Sep. 26, 2023

Related U.S. Application Data
(63) Continuation of application No. PCT/JP2022/012240, filed on Mar. 17, 2022.

(30) Foreign Application Priority Data
Mar. 31, 2021 (JP) 2021-061121
Jan. 19, 2022 (JP) 2022-006443

(51) Int. Cl. G01N 33/547; G01N 33/531; C08F 212/08

(52) U.S. Cl. CPC G01N 33/547; G01N 33/531; C08F 212/08; G01N 2474/00; C08F 2810/50

(57) ABSTRACT

A particle including a polymer which has a repeating unit derived from a vinyl-based monomer. The particle has a specific structure containing a carboxy group, and a surface of the particle contains a water-soluble polymer.

### CROSS-REFERENCE TO RELATED APPLICATIONS

This application is a Continuation of international Patent Application No. PCT/JP2022/012240, filed Mar. 17, 2022, which claims the benefit of Japanese Patent Application No. 2021-061121, filed Mar. 31, 2021 and the benefit of Japanese Patent Application No. 2022-006443, filed Jan. 19, 2022, all of which are hereby incorporated by reference herein in their entirety.

### TECHNICAL FIELD

The present invention relates to a particle used for a latex immunoagglutination method and a production method thereof.

### BACKGROUND ART

A latex immunoagglutination method has bees used as a method of measuring a substance to be measured in a biological sample (hereinafter, referred to as a specimen) in the field of a clinical test. According to this method, a dispersion liquid of particles to which an antibody or an antigen has been conjugated (bound) as a ligand is mixed with a specimen that may contain a substance to be measured (an antigen or an antibody). When a substance to be measured (an antigen or an antibody) is present in a specimen, since particles to which ligands have been conjugated undergo an agglutination reaction, the presence or absence of a substance to be measured can be specified or quantified by measuring the agglutination reaction as an amount of a change in scattered light intensity, transmitted light intensity, absorbance, or the like.

The particles used for the latex immunoagglutination method are required to improve detection sensitivity of the substance to be measured and to reduce a non-specific agglutination reaction. An increase in the amount of the ligands to be conjugated to the particles is considered to improve the detection sensitivity.

In recent years, research on purification or quantification of a substance to be measured has been conducted using affinity particles obtained by binding ligands having an affinity for the substance to be measured to particles. PTL 1 discloses latex particles having surfaces to which a carboxy group is introduced via an amino acid.

### CITATION LIST

### Patent Literature

PTL 1 International Publication No. WO 2007/063616

However, in the latex particles described in PTL 1, amines derived from amino acids which are present on the surfaces of the particles make the surface charges of the particles more positive. As a result, a non-specific agglutination reaction may occur. Further, there is a problem in that the amount of ligands to be conjugated to the carboxy groups on the surfaces of the particles is difficult to increase to a certain amount or greater.

Therefore, an object of the present invention is to provide particles capable of reducing the non-specific agglutination reaction and increasing the ligand conjugation amount.

### SUMMARY OF INVENTION

According to the present invention, there is provided a particle including a polymer which has a repeating unit derived from a vinyl-based monomer, in which the particle has a structure represented by Formula (1), and a surface of the particle contains a water-soluble polymer.

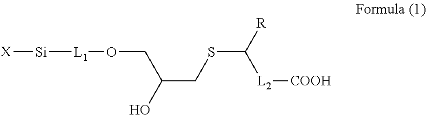

In Formula (1), X represents an atom contained in the polymer and bound to Formula (1), R represents a carboxyl group or a hydrogen atom, and L₁ and L₂ each independently represent an alkylene group having 1 or more and 15 or less carbon atoms or an oxyalkylene group having 1 or more and 15 or less carbon atoms.

According to the present invention, there is provided a method of producing a particle, the method including a first step of forming a particle containing a polymer by mixing a vinyl-based monomer, water, a radical polymerization initiator, and a water-soluble polymer, to obtain an aqueous dispersion liquid containing the particle, and a second step of forming a structure represented by Formula (1) on a surface of the particle by mixing a silane coupling agent containing a glycidyl group with a compound containing a mercapto group and a carboxy group in the aqueous dispersion liquid.

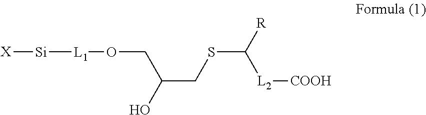

In Formula (1), X represents an atom contained in the polymer and bound to Si in Formula (1), R represents a carboxyl group or a hydrogen atom, and L₁ and L₂ each independently represent an alkylene group having 1 or more and 15 or less carbon atoms or an oxyalkylene group having 1 or more and 15 or less carbon atoms.

Further features of the present invention will become apparent from the following description of exemplary embodiments.

### DESCRIPTION OF EMBODIMENTS

Hereinafter, embodiments of the present invention will be described in detail, but the technical scope of the present invention is not limited to such embodiments.

A particle according to the present embodiment is a particle including a polymer which has a repeating unit derived from a vinyl-based monomer. The particle has a structure represented by Formula (1), and a surface of the particle contains a water-soluble polymer.

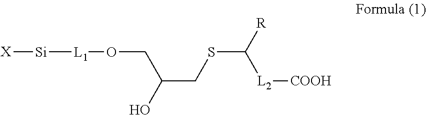

In Formula (1), X represents an atom contained in the polymer and bound to Si in Formula (1), R represents a carboxyl group or a hydrogen atom, and L₁ and L₂ each independently represent an alkylene group having 1 or more and 15 or less carbon atoms or an oxyalkylene group having 1 or more and 15 or less carbon atoms.

In the particle according to the present embodiment, the particle has a structure represented by Formula (1), and the surface of the particle contains a water-soluble polymer. Therefore, the surface charge of the particle is higher on a negative side as compared with the structure in which the surface of the particle contains an amine and contains no water-soluble polymer as described in PTL 1. Further, the particle according to the present embodiment contains a carboxy group with high hydrophilicity. As a result, the non-specific agglutination reaction is reduced. Here, when a ligand to be conjugated is an antibody, the antibody during the conjugation is in a cationic state. Therefore, the particle according to the present embodiment in which the surface charge of the particle is higher on a negative side as compared with the particle disclosed in PTL 1 is capable of increasing the ligand conjugation amount.

Further, the particle according to the present embodiment can be used for latex immunoagglutination. Specifically, the particle according to the present embodiment is a particle used for the latex immunoagglutination method. That is, since the particle according to the present embodiment can immobilize a ligand, and the obtained ligand-conjugated particle is bound to a target substance, the target substance can be measured by the latex immunoagglutination method. Since the ligand conjugation amount is large in the particle according to the present embodiment, the probability of the particle to be bound to the target substance is increased, and thus the target substance can be detected with high sensitivity by the latex immunoagglutination method.

The polymer of the particle according to the present embodiment may be a copolymer having a repeating unit derived from two or more kinds of vinyl-based monomers. Further, it is preferable that the copolymer have a repeating unit derived from a styrene-based monomer and a repeating unit derived from an organic silane compound containing a vinyl-based functional group.

### Monomer

The chemical structure of the repeating unit derived from a vinyl-based monomer according Co the present embodiment is not limited as long as the object of the present invention can be achieved. For example, a repeating unit derived from a styrene-based monomer, a repeating unit derived from a diene-based monomer, a repeating unit derived from a (meth)acrylic monomer, or a repeating unit derived from an organic silane compound containing a vinyl-based functional group can be used. In the present specification, “(meth)acryl” denotes acryl or methacryl.

### Repeating Unit Derived From Styrene-Based Monomer

The chemical structure of the repeating unit derived from a styrene-based monomer according to the present embodiment is not limited as long as the object of the present invention can be achieved. Examples thereof include repeating units derived from at least one monomer selected from the group consisting of styrene, α-methylstyrene, β-methylstyrene, o-methylstyrene, m-methylstyrene, p-methylstyrene, 2,4-dimethylstyrene, p-n-butylstyrene, p-tert-butylstyrene, p-n-hexylstyrene, p-n-octylstyrene, p-n-nonylstyrene, p-n-decylstyrene, p-n-dodecylstyrene, p-methoxystyrene, and p-phenylstyrene. In addition to these monomers, monomers having high compatibility with various monomers and containing a functional group with high hydrophilicity can be used alone or in combination of a plurality of kinds thereof. For example, a styrenesulfonic acid-based monomer (such as sodium p-styrene sulfonate) that has high compatibility with styrene and contains a sulfonic acid group as a functional group with high hydrophilicity can be used.

### Repeating Unit Derived From Diene-Based Monomer

The chemical structure of the repeating unit derived from a diene-based monomer according to the present embodiment is not limited as long as the object of the present invention can be achieved. Examples thereof include repeating units derived from at least one monomer selected from the group consisting of isoprene, butadiene, and chloroprene.

### Repeating Unit Derived From Methacrylic Monomer

The chemical structure of the repeating unit derived from a (meth)acrylic monomer according to the present embodiment is not limited as long as the object of the present invention can be achieved. Examples thereof include repeating units derived from at least one monomer selected from the group consisting of a (meth)acrylic; acid alkyl ester monomer and a (meth)acrylic acid alkoxy alkyl ester monomer. Examples of the repeating unit derived from the (meth)acrylic acid alkyl ester monomer include repeating units derived from at least one monomer selected from the group consisting of methyl (meth)acrylate, ethyl (meth))acrylate, n-propyl (meth)acrylate, isopropyl (meth)acrylate, n-butyl (meth)acrylate, isobutyl (meth)acrylate, n-hexyl (meth)acrylate 2-ethylhexyl (meth)acrylate, and cyclohexyl (meth)acrylate. Examples of the repeating unit derived from the (meth)acrylic acid alkoxy alkyl ester monomer include repeating units derived from at least one monomer selected from the group consisting of methoxymethyl (meth)acrylate, ethoxymethyl (meth)acrylate, 2-methoxyethyl (meth)acrylate, 2-ethoxyethyl (meth)acrylate, 2-propoxyethyl (meth)acrylate, 2-butoxyethyl (meth)acrylate, 3-methoxypropyl (meth)acrylate, and 4-methoxybutyl (meth)acrylate.

### Repeating Unit Derived From Organic Silane Compound Containing Vinyl-Based Functional Group

The chemical structure of the repeating unit derived from an organic silane compound containing a vinyl-based functional group according to the present embodiment is not limited as long as the object of the present invention can be achieved. Examples of the organic silane compound containing a vinyl-based functional group include repeating units derived from at least one monomer selected from the group consisting of vinyltrimethoxysilane, vinyltriethoxysilane, p-styryltrimethoxysilane, 3-methacryloxypropylmethyldimethoxysilane, 3-methacryloxypropyltrimethoxysilane, 3-methacryloxypropylmethyldiethoxysilane, 3-methacryloxypropyltriethoxysilane, and 3-acryloxypropyltrimethoxysilane.

### Radical Polymerization Initiator

As the radical polymerization initiator according to the present embodiment, an azo compound, an organic peroxide, or the like can be used. Specific examples thereof include 2,2′-azobis(isobutyronitrile), 2,2′-azobis(2,4-dimethylvaleronitrile), 2,2′-azobis(2-methylbutyronitrile), 4,4′-azobis(4-cyanovaleric acid), 2,2′-azobis(2-methylproponamidine) dihydrochloride, dimethyl 2,2′-azobis(2-methylpropionate), tart-butylhydroperoxide, benzoyl peroxide, ammonium persulfate (APS), sodium persulfate (NPS), and potassium persulfate (KPS).

### Water-Soluble Polymer

The water-soluble polymer in the present embodiment functions as a protective colloid during the synthesis of particles containing a polymer and contributes to controlling the particle diameter of polymer particles to be generated. Further, since the surface charges of the particles are higher on a negative side and the hydrophilicity is increased due to the presence of the water-soluble polymer, the non-specific agglutination can be reduced. As the water-soluble polymer in the present embodiment, at least one selected from the group consisting of polyacrylamide, polyvinyl alcohol, polyethylene oxide, and polyvinylpyrrolidone (PVP) can be used.

The molecular weight of the water-soluble polymer in the present embodiment is preferably 10000 or greater and 1000000 or less and more preferably 30000 or greater and 70000 or less. The reason for this is that when the molecular weight thereof is 10000 or greater, the effect as the protective colloid can be highly obtained and when the molecular weight thereof is 1000000 or less, the viscosity of an aqueous medium is not increased and thus the aqueous medium is unlikely to be handled with difficulty. Further, a part of such water-soluble polymers may adhere to the particle surfaces after the synthesis through physical adsorption, chemical adsorption, or the like.

### Structure of Particle Surface

The surface of the particle according to the present embodiment has a structure represented by Formula (1). Typically, an oxygen atom contained in the polymer and Si in Formula (1) form a siloxane bond. In this manner, the particle according to the present embodiment has a siloxane bond, and thus the structure represented by Formula (1) is firmly bound to the particle.

A silane coupling agent containing a glycidyl group and a compound containing a mercapto group and a carboxy group can be used to introduce the structure represented by Formula (1) to the particle. Examples of the silane coupling agent containing a glycidyl group include 3-glycidyloxypropyltrimethoxysilane and 3-glycidyloxypropyltriethoxysilane.

Examples or the compound containing a mercapto group and a carboxy group include mercaptoacetic acid, mercaptopropionic acid, and mercaptosuccinic acid.

### Details of Structure of Particle

It is preferable that the polymer contained in the particle according to the present embodiment have a repeating unit represented by Formula (2) and a repeating unit represented by Formula (3).

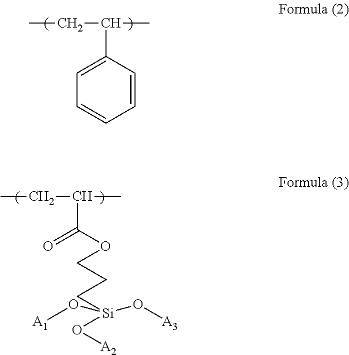

In Formula (3), A₁ to A₃ each independently represent any of H, CH₃, CH₂ CH₃, or a binding site to Si in Formula (3) via a single bond. The binding site to Si in Formula (3) via a single bond denotes a position where Si of Formula (3) in any one repeating unit and O of Formula (3) in another repeating unit are bound to each other when the polymer has a plurality of repeating units represented by Formula (3).

The particle according to the present embodiment has a high affinity and a high negative charge due to a combination of the polymer having repeating units represented by Formulae (2) and (3), the structure represented by Formula (1), and the water-soluble polymer of the particle surface, as described below. As a result, the non-specific agglutination reaction can be reduced, and the ligand conjugation amount can be increased. In this manner, high detection sensitivity with respect to the target substance is achieved when the particle is used for the late immunoagglutination method.

Further, the polymer in the present embodiment may further have a repeating unit represented by Formula (4).

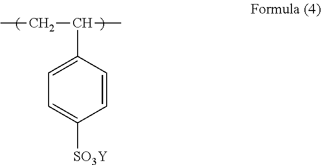

In Formula (4), Y represents any of H, Na, or K.

### Particle Diameter

The particle diameter of the particle according to the present embodiment is preferably 50 nm or greater and 1 μm or less and more preferably 50 nm or greater and 500 nm or less in terms of the number average particle diameter in water.

When the particle diameter thereof is 50 nm or greater and 500 nm or less, the handleability in a centrifugal operation is excellent, and the size of the non-surface area which is a feature of the particle stands out. The average particle diameter in the present embodiment can be measured by a dynamic light scattering method. Specifically, the dispersion liquid of the polymer particles is measured by the dynamic light scattering method, the light intensity to be obtained is converted to the number distribution, and the average value thereof can be defined as the particle diameter.

### Ligand

In the present embodiment, a ligand can be bound to the carboxy group of the particle according to the present embodiment. A particle obtained by binding a ligand to the particle according to the present embodiment will be referred to as a ligand-conjugated particle. Further, binding of a ligand to the particle according to the present embodiment will be referred to as ligand conjugation below.

The ligand in the present embodiment denotes a compound specifically bound to a receptor of a specific target substance. A site where the ligand is bound to the target substance is determined and has a selectively or specifically high affinity. Examples of the ligand include an antigen and an antibody, an enzyme protein and a substrate thereof, a signal substance such as a hormone or a neurotransmitter and a receptor thereof, and a nucleic acid, but the ligand of the present invention is not limited thereto. The conjugated particle for latex immunoagglutination in the present embodiment denotes a conjugated particle for latex immunoagglutination having a high affinity selectively or specifically for the target substance.

In the present embodiment, a method of carrying out a chemical reaction of chemically immobilizing a ligand and a carboxy group contained in the particle according to the present embodiment can be performed by applying a known method of the related art within a range where the object of the present invention can be achieved. For example, a carbodiimide-mediated reaction or an NHS ester activation reaction are commonly used chemical reactions. However, the method of carrying out a chemical reaction of chemically immobilizing a ligand and a carboxy group in the present invention is not limited thereto.

In the ligand-conjugated particles in the present embodiment, the carboxy groups remaining without ligands being bound thereto may be actively esterified, and hydrophilic molecules may be bound thereto. The treatment is typically referred to as inactivation of an active ester, a blocking treatment of a carboxy group, a masking treatment, or the and is performed for reducing non-specific adsorption of proteins on the carboxy groups and improving the dispersion stability of the ligand-conjugated particles. In the present embodiment, it is preferable that the hydrophilic molecules be polyethylene glycol (PEG) or trishydroxymethylaminomethane (Tris). PEG is particularly preferable from the viewpoint of greatly reducing adsorption of proteins on the particles. When the inactivation of the active ester is performed by using PEG as described in examples below, the molecular weight of PEG is important, and the antigen-antibody reaction can be disturbed in a case where the molecular weight thereof is large. Therefore, the molecular weight of PEG is preferably 350 or greater and 5000 or less and particularly preferably 1000 or greater and 2000 or less. As PEG in the present embodiment, PEG containing a functional group that is reactive to a carboxy group or an active ester, for example, PEG containing an amino group is preferable and polyethylene glycol containing a primary amine is particularly preferable. The polyethylene glycol may be a linear polymer or a branched polymer. Tris is represented by Formula (5), and an example of PEG is represented by Formulae (6) and (7). Further, n in Formulae (4) and (5) represents an integer of 1 or greater indicating the number of oxyethylene units.

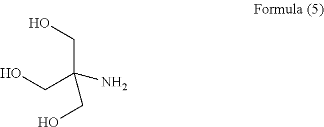

CH₂O—(CH₂CH₂O)n-CH₂CH₂NH₂ Formula (6)

CH₃O—(CH₂CH₂O)n-CH₂CH₂CH₂NH₂ Formula (7)

The amount of the ligand to be bound is also an important factor, and the reactivity to an antigen and an antibody is degraded in a case where the ligand binding amount (also referred to as an immobilization amount) is small, which is not preferable. When the ligand binding amount is large in the reaction, the dispersability of the ligand-conjugated particles may be degraded. The ligand binding amount varies depending on the particle diameter, but is preferably 1 μg or greater and 500 μg or less and particularly preferably 10 μg or greater and 200 μg or less with respect to 1 mg of the particles when the average particle diameter is about 200 nm.

The ligand-conjugated particles for latex immunoagglutination in the present embodiment use an antibody or an antigen as a ligand and can be preferably applied to a latex immunoagglutination measuring method that has been widely used in the field of the clinical test, biochemical research, and the like. When typical particles are app led to the latex immunoagglutination measuring method, there is a problem in that antigens (antibodies) as target substances, foreign substances in the serum, and the like are non-specifically adsorbed on the surfaces of the particles, the non-specific adsorption leads to unintended detection of agglutination of particles, and thus accurate measurement is impaired. Therefore, for the purpose of reducing a deceptive noise, biological substances such as albumin are typically used by being applied as a blocking agent to particles to reduce non-specific adsorption on the surfaces of particles. However, since the properties of such biological substances slightly vary depending on the lot, the particles coated with these biological substances differ in ability to reduce non-specific adsorption for respective coating treatments. Therefore, there is a problem of stably supplying particles having the same level of ability to suppress non-specific adsorption. Further, the biological substances applied to the surfaces of the particles exhibit hydrophobicity due to denaturation in some cases and does not necessarily have an excellent ability to reduce non-specific adsorption. Further, biological contamination may also be a problem. PTL 1 discloses posted-coated ligand-conjugated particles for latex immunoagglutination which are used to reduce the non-specific reaction. However, the post-coating agent is water-soluble and applied to the surface of the particle through physical adsorption, and thus there is a problem of essential liberation due to dilution. The ligand-conjugated particles according to the present embodiment are highly hydrophilized particles having an enhanced ability to reduce the non-specific adsorption. Therefore, the above-described problem can be solved without carrying out post-coating with albumin or the like.

### Test Reagent

A test reagent for in vitro diagnosis according to the present embodiment contains the ligand-conjugated particles according to the present embodiment and a dispersion medium. The amount of the ligand-conjugated particles contained in the test reagent according to the present embodiment is preferably in a range of 0.0011 by mass to 20% by mass and more preferably in a range of 0.01% by mass to 10% by mass. The reagent of the present invention may contain a third substance such as a solvent or a blocking agent within a range where the object of the present invention can be achieved in addition to the ligand-conjugated particles for latex immunoagglutination of the present invention. The third substance such as a solvent or a blocking agent may be used in combination of two or more kinds thereof. Examples of the solvent used in the present invention include various buffer solutions such as a phosphate buffer solution, a glycine buffer solution, a Good's buffer solution, a tris buffer solution, and an ammonia buffer solution, but the solvent contained in the reagent of the present invention is not limited thereto.

A test kit for in vitro diagnosis according to the present embodiment includes the test reagent according to the present embodiment and a housing including the test reagent. It is preferable that the test kit according to the present embodiment further include a reaction buffer solution (hereinafter, referred to as a reagent 2) containing albumin in addition to the test reagent (hereinafter, a reagent 1) according to the present embodiment. Examples of the albumin include serum albumin, which may be protease-treated. The amount of albumin contained in the reagent 2 is in a range of 0.001% by mass to 5% by mass as a guideline, but the test kit according to the present embodiment is not limited thereto. Both or any one of the reagent 1 and the reagent 2 may contain a sensitizer for latex immunoagglutination measurement. Examples of the sensitizer for latex inmmunoagglutination measurement include polyethylene glycol, polyvinyl alcohol, polyvinylpyrrolidone, and polyalginic acid, but the sensitizer is not limited thereto. Both or any one of the reagent 1 and the reagent 2 may contain a surfactant. Since the surfactant has an effect of stabilizing particles and proteins, polyoxyethylene sorbitan monolaurate, poly(oxyethylene)octyl phenyl ether, or the like is suitably used, for example. Further, the test kit according to the present embodiment may include a positive control, a negative control, and a serum diluent in addition to the reagent 1 and the reagent 2. As a medium of the positive control or the negative control, serum containing no measurable target substance, physiological saline, or a solvent may be used.

### Detection Method

A method of detecting a target substance in a specimen using the latex immunoagglutination method according to the present embodiment is performed by mixing the ligand-conjugated particles for latex immunoagglutination according to the present embodiment with a specimen that may contain a target substance. Further, it is preferable that the ligand-conjugated particles for latex immunoagglutination according to the present embodiment be mixed with the specimen in a pH range of 3.0 to 11.0. Further, the mixing temperature is in a range of 20° C. to 50° C., and the mixing time is in a range of 10 seconds to 30 minutes. Further, the concentration of the ligand-conjugated particles for latex immunoagglutination according to the present embodiment in the detection method according to the present embodiment is preferably in a range of 0.001% by mass to 5% by mass and more preferably in a range of 0.01% by mass to 1% by mass in the reaction system. The detection method according to the present embodiment is performed by optically detecting the agglutination reaction occurring as a result of the mixing of the ligand-conjugated particles for latex immunoagglutination according to the present embodiment with the specimen. Specifically, when the agglutination reaction is optically detected, the target substance in the specimen is detected, and thus the concentration of the target substance can also be measured. A method of optically detecting the agglutination reaction may be performed by measuring the amount of a change in values of the scattered light intensity, the transmitted light intensity, the absorbance, or the like using an optical device capable of detecting these values.

### Production Method

A method of producing the particles according to the present embodiment of the present invention will be described. The method of producing the particles according to the present embodiment includes at least a first step and a second step described below.

### First Step

The first step is a step of forming particles (granular copolymer) containing a copolymer by mixing a vinyl-based monomer, water, a radical polymerization initiator, and a water-soluble polymer, to obtain an aqueous dispersion liquid containing the particles.

### Second Step

The second step is a step of forming a structure represented by Formula (1) on surfaces of the particles by mixing a silane coupling agent containing a glycidyl group with a compound containing a mercapto group and a carboxy group in the aqueous dispersion liquid. The details will be described below.

In the first step in the present embodiment, a granular copolymer is formed by mixing a vinyl-based monomer, an organic silane compound containing a vinyl-based functional group, a water-soluble polymer, water, and a radical polymerization initiator, to obtain an aqueous dispersion liquid. For example, styrene, 3-methacryloxypropyltrimethoxysilane, and polyvinylpyrrolidone can be used as the vinyl-based monomer, the organic silane compound containing a vinyl-based functional group, and the water-soluble polymer.

The first step in the present embodiment includes a step of mixing, for example, a silane coupling agent containing a glycidyl group, and a compound containing a mercapto group and a carboxy group in an aqueous dispersion liquid of the polymer. In this manner, an aqueous dispersion liquid containing the particles in which the particle surfaces have the structure represented by Formula (1), that is, a carboxy group is introduced to the particles is obtained. For example, 3-glycidyloxypropyltrimethoxysilane and mercaptosuccinic acid can be used as the silane coupling agent containing a glycidyl group and the compound containing a mercapto group and a carboxy group.

Here, the aqueous dispersion liquid of the granular copolymer may be mixed with the compound containing a mercapto group and a carboxy group after the aqueous dispersion liquid is mixed with the silane coupling agent containing a glycidyl group in advance and the particle surface is modified with the glycidyl group. Further, the particle surface may be modified with a carboxy group by mixing the silane coupling agent containing a glycidyl group with the compound containing a mercapto group and a carboxy group in advance and mixing the mixture with the aqueous dispersion liquid of the granular copolymer.

### EXAMPLES

Hereinafter, the present invention will be described in detail with reference to examples, but the present invention is not to these examples.

### Example 1 Synthesis of Particles

160 g of a 2-morpholinoethanesulfonic acid (MES) buffer solution (manufactured by Tokyo Chemical Industry Co., Ltd., 50 mM, pH of 7.0) was poured into a 200 mL flask, and 1.3 g of polyvinylpyrrolidone K-30 (manufactured by Kishida Chemical Co., Ltd., molecular weight of 40000) was dissolved therein. Next, 4.1 g of 3-methacryloxypropyltrimethoxysilane (trade name: LS-3380, manufactured by Shin-Etsu Chemical Co., Ltd.) and 12.9 g of styrene (manufactured by Kishida Chemical Co., Ltd.) were added thereto, and the solution was stirred for 10 minutes while nitrogen was blown at room temperature. Thereafter, the emulsion in the flask was heated to 70° C. in an oil bath. A solution obtained by dissolving 0.4 g of potassium peroxodisulfate (manufactured by Wako Pure Chemical industries, Ltd.) in 10 mL of pure water was added to the emulsion heated to 70° C. The solution was stirred at 70° C. for 7 hours and cooled to room temperature, thereby obtaining a dispersion liquid of particles (hereinafter, referred to as SA1).

Further, the dispersion liquid of SA1 was centrifuged by a centrifugal separator to recover SA1, and the supernatant was disposed of. The recovered SA1 was redispersed in pure water and centrifuged again by a centrifugal separator. The recovery of SAI using a centrifugal separator and the redispersion in pure water were repeated four times.

The average particle diameter of SA1 was evaluated by DLS, and the value was 161 nm.

Next, 3-glycidyloxypropyltrimethoxysilane and the compound containing a mercapto group and a carboxy group were mixed with SA1 as listed in Table 1, and the mixture was allowed to react at 60° C. for 24 hours. When acid base titration was performed while the electrical conductivity of the particle solution was measured, the amount of the carboxy group of the particles can be calculated from the titration curve. The amounts of the carboxy groups of the prepared particles are collectively listed in Table 1.

TABLE 1

| | 3-Glycidyl- | Compound | | Amount of |
|---|---|---|---|---|
| | oxypropyl- | containing mercapto group | | carboxy |
| | trimethoxysilane | and carboxy group | | group |
| Particles | (mL) | Name | Amount (mg) | (nmol/mg) |
| SAS1 | 0.02 | Mercapto- | 20 | 20 |
| | | succinic acid | | |
| SAS2 | 0.02 | Mercapto- | 20 | 20 |
| | | propionic acid | | |
| SAS3 | 0.6 | Mercapto- | 600 | 26 |
| | | succinic acid | | |
| SAS4 | 0.6 | Mercapto- | 600 | 23 |
| | | propionic acid | | |

### Comparative Example 1 Modification With Carboxy Group Using Compound Containing Amino Group and Carboxy Group

Particles (hereinafter, referred to as SAN1) were prepared by the same method as the method for SAS1 except that the compound containing a mercapto group and a carboxy group described in Example 1 was changed to a compound containing an amino group and a carboxy group. As a result of calculation of the amount of the carboxy group, the amount thereof in a case of SAN1 was 13 nmol/mg, which was found to be smaller than that of the particles described in Example 1. One of the reasons for this is considered to be the mercapto group having more excellent reactivity to the glycidyl group. Another reason for this is considered to be the carboxy group reacted with the glycidyl group in SAN1.

### Comparative Example 2 Polystyrene Particles

In the comparative example, IMMUTEX (manufactured by JSR Corporation, P0113, 188 nm) serving as polystyrene particles containing a carboxy group was used. IMMUTEX diluted to a 0.1 wt % solution with ion exchange water was used.

### Example 2 Evaluation of Non-Specific Agglutination Reaction (Non-Specific Agglutination) of Particles

The non-specific agglutination of particles was evaluated by an immune-nephelometry. The immune-nephelometry is a method of measuring non-specifically occurring agglutination of particles with an absorbance meter using the turbidity as an index by bringing human serum and the particles into contact with each other. When non-specific agglutination occurs, the absorbance is increased. The absorbance was measured by injecting a sample into a plastic cell (minimum sample volume of 70 μL) and setting the optical path length to 10 mm using an ultraviolet visible spectrophotometer (GeneQuant 1300, Ge HealthCare). The specific measuring method is as follows.

5 μL of human serum and 50 μL of an R1 buffer solution were mixed in a plastic cell and warmed at 37° C. for 5 minutes. 50 μL of a particle dispersion solution (particle concentration of 0.1 wt %) was added to the R1 buffer solution (55 μL) containing human serum, and the solution was quickly pipetted while being careful to prevent entrance of air bubbles and used as a sample. The absorbance of the sample at 572 nm was read and defined as Abs1. The sample was warmed at 37° C. for 5 minutes, and the absorbance of the sample at 572 nm was read and defined as Abs2. A value obtained by subtracting Abs1 from Abs2 and multiplying the subtracted value by 10000 was defined as a value of ΔOD×10000. When the value of ΔOD×10000 was 2000 or greater, it was determined that non-specific agglutination had occurred.

TABLE 2

| | Evaluation of reduction in | |
|---|---|---|
| | non-specific agglutination | |
| | | Presence or |
| | | absence of |
| | Change in | non-specific |
| Particles | absorbance | agglutination |
| SAS1 | 20 | Absent |
| SAS2 | −100 | Absent |
| SAS3 | 20 | Absent |
| SAS4 | 30 | Absent |
| P0113 | 5000 or greater | Present |
| (Comparative Example 2) | | |

The results are listed in Table 2. The ΔOD×10000 of the particles in the present example was 2000 or less, and the non-specific reaction was not observed. Meanwhile, the ΔOD×10000 of IMMUTEX serving as polystyrene particles that had not been coated was 10000 or greater. It was found that since the absorbance of the dispersion liquid was considered to be increased due to the occurrence of agglutination between particles as a result of non-specific adsorption of the substance contained in human serum on the particles in the dispersion liquid, IMMUTEX was non-specifically agglutinated by the serum. It was confirmed that the particles of the present example were capable of further reducing non-specific adsorption as compared with commercially available polystyrene particles.

### Example 3 Preparation of Antibody-Conjugated Particles

0.1 mL (1 mg of particles) of the dispersion liquid (solution with concentration of 1.0% by mass, 10 mg/mL) containing the particles of the present example was transferred to microtubes (volume of 1.5 mL). 0.12 mL of an activation buffer solution (25 mM MES buffer solution, pH of 6.0) was added to the microtubes and centrifuged at 4° C. and 15000 rpm (20400 g) for 20 minutes. After the centrifugation, the supernatant was disposed of using a pipette. Next, 0.12 mL of an activation buffer solution was added thereto and dispersed by ultrasonic waves using an ultrasonic cleaner (trade name: MODEL VS-100III, AS ONE Three Frequency Ultrasonic Cleaner, manufactured by AS ONE Corporation, 28 kHz). Next, the solution was centrifuged at 4° C. and 15000 rpm (20400 g) for 20 minutes. The supernatant was disposed of using a pipette, and 0.12 mL of an activation buffer solution was added thereto and dispersed by ultrasonic waves. Next, the solution was centrifuged at 4° C. and 15000 rpm (20400 g) for 20 minutes, and the supernatant was disposed of with a pipette. Next, 60 μL of a WSC solution (solution obtained by dissolving 50 mg of WSC in 1 mL of the activation buffer solution) and 60 μL of an N-hydroxysulfosuccinic acid imide (Sulfa NHS) solution (solution obtained by dissolving 50 mg of Sulfo NHS in 1 mL of an activation buffer solution) were added thereto. After the addition, the solution was dispersed by ultrasonic waves. Further, the solution was stirred at room temperature for 30 minutes so that the carboxy groups of the particles were converted to active esters.

Next, the dispersion liquid was centrifuged at 4° C. and 15000 rpm (20400 g) for 20 minutes, and the supernatant was disposed of with a pipette. 0.2 mL of an immobilization buffer solution (25 mM MES buffer solution, pH of 5.0) was added thereto and dispersed by ultrasonic waves. The solution was centrifuged at 4° C. and 15000 rpm (20400 g) for 20 minutes, and the supernatant was disposed of with a pipette. 50 μL of an immobilization buffer solution per 1 mg of particles was added thereto, and particles with activated carboxy groups were dispersed by ultrasonic waves.

An anti-human CRP antibody (polyclonal antibody) was diluted to 100 μg/50 μL, with an immobilization buffer solution (referred to as an antibody solution). 50 μL of the antibody solution was added to 50 μL of a solution (containing 1 mg of particles) containing particles with activated carboxy groups, and the particles were dispersed by ultrasonic waves. The charged amount of the antibody was set to 100 μg per 1 mg of the particles (100 μg/mg). The tubes were stirred at room temperature for 60 minutes so that the antibodies were immobilized to the carboxy groups of the particles. Next, the solution was centrifuged at 4° C. and 15000 rpm (20400 g) for 20 minutes, and the supernatant was disposed of with a pipette.

0.24 mL of an active ester inactivation buffer solution (1 M Tris buffer solution containing 0.1% of Tween (registered trademark) 20 with a pH of 8.0) containing tris(hydroxymethyl)aminomethane (Tris) was added thereto and dispersed by ultrasonic waves. The solution was stirred at room temperature for 1 hour so that Tris was bound to the remaining activated esters, and the solution was allowed to stand overnight at 4° C.

Next, the solution was centrifuged at 4° C. and 15000 rpm (20400 g) for 20 minutes, and the supernatant was disposed of with a pipette. 0.2 mL of a cleaning and storing buffer solution (10 mM 2-[4-(2-hydroxyethyl)-1-piperazinyl]-ethanesulfonic acid (HEPES) buffer solution, pH of 7.9) was added thereto and dispersed by ultrasonic waves. A cleaning operation with 0.2 mL of the cleaning and storing buffer solution was repeated twice, and 1.0 mL of the cleaning and storing buffer solution was added thereto and dispersed by ultrasonic waves. In the above-described step, almost no loss of particles was found, and thus the final concentration of the antibody-conjugated particles was 0.1% by mass (1 mg/mL). The particles were stored in a refrigerator and redispersed by ultrasonic waves when used.

### Comparative Example 3 Conjugation of Antibodies to Particles in Comparative Example

In the comparative example, antibodies were conjugated to SAN1 described above and polystyrene particles (IMMUTEX, P0113, manufactured by JSR Corporation). Antibody-conjugated particles of the comparative example were obtained by the same experiment operation as in Example 3 except that the particles of Example 3 were changed.

### Example 4 Measurement of Antibody Conjugation Efficiency

It was confirmed by protein quantification that the antibodies were conjugated (immobilized) to the particles. Specifically, the conjugation was confirmed by a method of reacting the antibody-conjugated particles with a BCA reagent. First, 25 μL (particle amount of 25 μg) of a dispersion liquid (0.1% solution) of the antibody-conjugated particles was sorted. 7 mL of an A liquid and 140 μL of a B liquid in BCA Protein Assay Kit (Wako Pure Chemical Industries, Ltd.) were mixed to obtain an AB liquid. 200 μL of the AB liquid was added to the particle solution (25 μL), and the solution was incubated at 60° C. for 30 minutes.

The solution was centrifuged at 4° C. and 15000 rpm (20400 g) for 5 minutes, and 200 μL of the supernatant was recovered with a pipette. The absorbance at 562 nm was measured with a standard sample (multiple antibodies in a range of 0 to 200 μg/mL with 10 mM HEPES) using a multimode microplate reader (Synergy MX, BioTek). The amount of antibodies was calculated from the standard curve. The amount of antibodies conjugated to the particles (antibody binding amount (antibody immobilization amount) (μg/mg) per weight of particles) was determined by dividing the calculated amount of antibodies by the weight of particles (0.025 mg here). The conjugation efficiency was determined from the charged amount of antibodies. The results are listed in Table 3. It was found that the antibody-conjugated particles of the present example had higher conjugation efficiency than that of the comparative examples.

The high conjugation efficiency of the particles in the present example will be described. In the particles of the present example, the carboxy group was positioned in the vicinity of the surface, and the particle surface was coated with a water-soluble polymer (PVP).

That is, the surface charges were uniformly negative, and the hydrophilicity was extremely high. The hydrophilic surface exhibits a high ability to suppress non-specific adsorption. The antibodies were in a cationic state during the conjugation and electrostatically attracted to the region of the carboxy groups of the particles having a negative surface. As a result, the antibodies were concentrated on the particle surfaces, and the reaction between the antibodies and the carboxy groups on the particle surfaces was greatly promoted. As a result, the rate of the antibodies conjugated to the particles was considered to be improved. Since the NHS activated ester of the carboxy group was rapidly hydrolyzed in water, the action of concentrating the antibodies on the particle surfaces was an important process.

In the SANI particles of the comparative examples, carboxy groups and amino groups were positioned in the vicinity of the particle surfaces, and thus the amount of negative surface charges was smaller than that of the particles of the present example. As a result, the rate of the antibodies conjugated to the particles was considered to be decreased.

Meanwhile, the particle surfaces are hydrophobic in commercially available polystyrene, and thus physical adsorption of the antibody to a hydrophobic portion other than the region of the carboxy group occurs. The physical adsorption results in repetition of desorption and adsorption, and thus the conjugation efficiency of commercially available polystyrene particles was considered to be decreased.

TABLE 3

| Antibody-conjugated | Raw material | Conjugation |
|---|---|---|
| particles | particles | efficiency (%) |
| SAS1-Ab100 | SAS1 | 54 |
| SAS2-Ab100 | SAS2 | 64 |
| SAS3-Ab100 | SAS3 | 75 |
| SAS4-Ab100 | SAS4 | 93 |
| SAN1-Ab100 | SAN1 | 27 |
| (Comparative Example 3) | | |
| P0113-Ab100 | P0113 | 8 |
| (Comparative Example 3) | | |

### Example 5 Evaluation of Sensitivity of Antibody-Conjugated Particles to Human-CRP Antigen

The sensitivity of the antibody-conjugated particles was evaluated by the latex immunoagglutination method. Specifically, the evaluation was performed by a method of reacting the antibody-conjugated particles with antigens to form an agglutinate of an immune complex, irradiating the agglutinate with light, and measuring the attenuation (absorbance) of irradiation light due to scattering using an absorbance meter. The proportion of the agglutinate was increased depending on the amount of antigens contained in the specimen, and the absorbance was increased. It was desirable that the amount of an increase (described in ΔOD×10000) in absorbance at a predetermined PSA concentration was large. The absorbance was measured by injecting a sample into a plastic cell and setting the optical path length to 10 mm using an ultraviolet visible spectrophotometer (GeneQuant 1300, Ge HealthCare). The specific measuring method is as follows.

Specifically, 1μL of a CRP solution (CRP concentration of 0 μg/mL or 160 μg/mL) was used as a sample, this sample and 50 μL of an R1 buffer solution were mixed in a plastic cell and warmed at 37° C. for 5 minutes. 50 μL of a dispersion solution of the antibody-conjugated particles (particle concentration of 0.1 wt %, 10 mM HEPES, pH of 7.9, 0.01 wt % Tween (registered trademark) 20) was added to the R1 buffer solution (51 μL) containing CRP, and the solution was quickly pipetted while being careful to prevent entrance of air bubbles and used as a sample. The absorbance of the sample at 572 nm was read and defined as Abs1. The sample was warmed at 37° C. for 5 minutes, and the absorbance of the sample at 572 nm was read and defined as Abs2. A value obtained by subtracting Abs1 from Abs2 and multiplying the subtracted value by 10000 was defined as a value of ΔOD×10000.

The results are listed in Table 4. In the antibody-conjugated particles of the present example, an increase in ΔOD×10000 in the presence of CRP was observed. It was found that this increase was the result of binding of the antibody-conjugated particles to CRP which is an antigen, to form particle agglutinates, and the antibody-conjugated particles functioned as particles used for the latex immunoagglutination method.

TABLE 4

| | Sensitivity (ΔOD × 10000) | |
|---|---|---|
| Antibody-conjugated | CRP concentration | CRP concentration |
| particles | 0 ug/mL | 160 ug/mL |
| SAS3-Ab100 | −60 | 8100 |
| SAS4-Ab100 | −40 | 10890 |
| SAN1-Ab100 | 40 | 220 |
| (Comparative Example 3) | | |

### Example 6 Synthesis of Particles Using Styrenesulfonic Acid-Based Monomer

160 g of an MES buffer solution (manufactured by Tokyo Chemical Industry Co., Ltd., 50 mM, pH of 7.0) was poured into a 200 mL flask, and 2 g of polyvinylpyrrolidone K-30 (manufactured by Kishida Chemical Co., Ltd., molecular weight of 40000) was dissolved therein. Next, 2 g of 3-methacryloxypropyltrimethoxysilane (trade name: LS-3380, manufactured by Shin-Etsu Chemical Co., Ltd.), 6.4 g of styrene (manufactured by Kishida Chemical Co., Ltd.), and 0.1 g of sodium p-styrene sulfonate (manufactured by Tokyo Chemical Industry Co., Ltd.) were added thereto, and the solution was stirred for 10 minutes while nitrogen was blown at room temperature. Thereafter, the emulsion in the flask was heated to 70° C. in an oil bath. A solution obtained by dissolving 0.5 g of potassium peroxodisulfate (manufactured by Wako Pure Chemical Industries, Ltd.) in 10 mL of pure water was added to the emulsion heated to 70° C. The solution was stirred at 70° C. for 7 hours and cooled to room temperature, thereby, obtaining a dispersion liquid of particles (hereinafter, referred to as SA2).

Further, the dispersion liquid of SA2 was centrifuged by a centrifugal separator to recover SA2, and the supernatant was disposed of. The recovered SA2 was redispersed in pure water and centrifuged again by a centrifugal separator. The recovery of SA2 using a centrifugal separator and the redispersion in pure water were repeated four times.

The average particle diameter of SA2 was evaluated by DLS, and the value was 72 nm.

Next, 3-glycidyloxypropyltrimethoxysilane and the compound containing a mercapto group and a carboxy group were mixed with SA2 as listed in Table 5, and the mixture was allowed to react at 70° C. for 24 hours. The amounts of the carboxy groups of the prepared particles (SAS5) are collectively listed in Table 5. The average particle diameter of SASS was evaluated by DLS, and the value was 76 nm. That is, an increase in the particle diameter and the presence of the carboxy group were similarly confirmed even in the particles formed by using a styrenesulfonic acid-based monomer.

TABLE 5

| | 3-Glycidyloxypropyl- | Compound containing | | COOH |
|---|---|---|---|---|
| | trimethoxysilane | COOH | | amount |
| Particles | (mL) | Name | mg | (nmol/mg) |
| SAS5 | 0.6 | Mercaptosuccinic | 600 | 43 |
| | | acid | | |

The present invention is not limited to the above-described embodiments, and various changes and modifications can be made within a range not departing from the spirit and the scope of the present invention. Therefore, the following claims are appended to disclose the scope of the present invention.

According to the present invention, it is possible to provide particles capable of reducing the non-specific agglutination reaction and increasing the ligand conjugation amount.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A particle comprising:
a polymer which has a repeating unit derived from a vinyl-based monomer,
wherein the particle has a structure represented by Formula (1), and
a surface of the particle contains a water-soluble polymer, 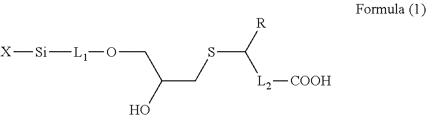
in Formula (1), X represents an atom contained in the polymer and bound to Si in Formula (1), R represents a carboxyl group or a hydrogen atom, and L₁ and L₂ each independently represent an alkylene group having 1 or more and 15 or less carbon atoms or an oxyalkylene group having 1 or more and 15 or less carbon atoms.

2. The particle according to claim 1, wherein the polymer has a repeating unit derived from a styrene-based monomer and a repeating unit derived from an organic silane compound containing a vinyl-based functional group.

3. The particle according to claim 1, wherein the polymer has a repeating unit represented by Formula (2) and a repeating unit represented by Formula (3), 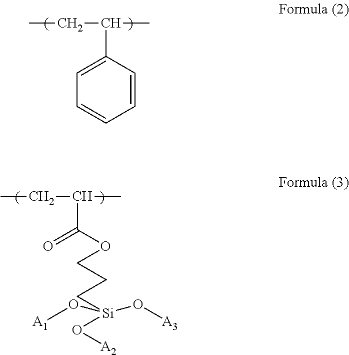
in Formula (3), A₁ to A₃ each independently represent any of H, CH₃, CH₂CH₃, or a binding site to Si in Formula (3) via a single bond.

4. The particle according to claim 1, wherein the polymer further has a repeating unit represented by Formula (4), 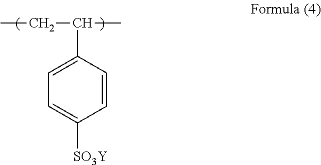
in Formula (4), Y represents any of H, Na, or K.

5. The particle according to claim 1, wherein the particle has a particle diameter of 50 nm or greater and 500 nm or less.

6. The particle according to claim 1, wherein the water-soluble polymer is at least one selected from the group consisting of polyacrylamide, polyvinyl alcohol, polyethylene oxide, and polyvinylpyrrolidone.

7. The particle according to claim 1, wherein the particle has a siloxane bond.

8. A ligand-conjugated particle having a ligand bound to a carboxy group contained in the particle according to claim 1.

9. The ligand-conjugated particle according to claim 8, wherein an amount of the ligand bound to the particle is 1 μg or greater and 500 μg or less with respect to 1 mg of the particle.

10. A test reagent for in vitro diagnosis, comprising:
the ligand-conjugated particle according to claim 8; and
a dispersion medium which disperses the ligand-conjugated particle.

11. The test reagent according to claim 10, wherein the ligand is an antibody or an antigen, and
the test reagent is used for detecting an antigen or an antibody in a specimen by a latex immunoagglutination method.

12. A test kit for in vitro diagnosis, comprising:
the test reagent according to claim 10; and
a housing which includes the test reagent.

13. A method of producing a particle, the method comprising:
a first step of forming a particle containing a polymer by mixing a vinyl-based monomer, water, a radical polymerization initiator, and a water-soluble polymer, to obtain an aqueous dispersion liquid containing the particle; and
a second step of forming a structure represented by Formula (1) on a surface of the particle by mixing a silane coupling agent containing a glycidyl group with a compound containing a mercapto group and a carboxy group in the aqueous dispersion liquid, 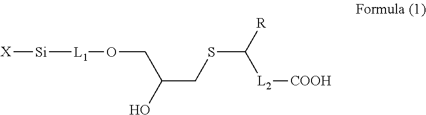
in Formula (1), X represents an atom contained in the polymer and bound to Si in Formula (1), R represents a carboxyl group or a hydrogen atom, and L₁ and L₂ each independently represent an alkylene group having 1 or more and 15 or less carbon atoms or an oxyalkylene group having 1 or more and 15 or less carbon atoms.
